(19) United States
(12) Patent Application Publication
(10) Pub. No.: US 2004/0210166 A1
(43) Pub. Date: Oct. 21, 2004

(54) Apparatus and method for detecting finger-motion

(75) Inventors: Byung-Seok Soh, Suwon-city (KR); Sang-Goog Lee, Anyang-city (KR); Ji-Hun Koo, Yongin-city (KR); Joon-Kee Cho, Yongin-city (KR)

Correspondence Address: SUGHRUE MION, PLLC, 2100 PENNSYLVANIA AVENUE, N.W., SUITE 800, WASHINGTON, DC, 20037, US

(73) Assignee: SAMSUNG ELECTRONICS CO., LTD.

(21) Appl. No.: 10/735,906

(22) Filed: Dec. 16, 2003

(30) Foreign Application Priority Data
Apr. 18, 2003 (KR) P2003-24777

(51) Int. Cl. A61B 5/103

(52) U.S. Cl. 600/595; 128/903

(57) ABSTRACT

Provided is an apparatus and method for detecting finger-motion in a wireless manner through a small finger-motion detecting apparatus without an additional battery, which is inexpensive and convenient to use. The apparatus includes a finger-motion signal receiving unit, which outputs a wireless power signal and receives and reads a wireless finger-motion signal to determine the corresponding finger-motion, a finger-motion signal transmitting unit, which generates a predetermined amount of power using the wireless power signal, receives a finger-motion signal corresponding to finger-motion using the predetermined amount of power, modulates the finger-motion signal into a finger-motion signal having a predetermined frequency, and outputs the modulated finger-motion signal in a wireless manner, and a finger-motion detecting unit, which detects whether or not finger-motion exists and generates a finger-motion signal corresponding to the finger-motion.

This application claims the priority of Korean Patent Application No. 2003-24777, filed on Apr. 18, 2003, in the Korean Intellectual Property Office, the disclosure of which is incorporated herein in its entirety by reference.

### BACKGROUND OF THE INVENTION

1. Field of the Invention

The present invention relates to an apparatus and method for detecting finger-motion, and more particularly, to a wireless finger-motion detecting apparatus and method, which can be used in a wearable input device.

2. Description of the Related Art

FIGS. 1A and 1B are diagrams of a conventional wearable input device. As shown in FIGS. 1A and 1B, the conventional wearable input device includes sensors, which are respectively installed on fingers to detect the motion of the fingers. The sensors are connected in a wired manner to provide driving power to the sensors and transmit signals detected by the sensors. Since the conventional wearable finger-motion detecting device receives signals detected by the sensors in a wired manner, the users wearing the device are restricted in motion.

To solve these problems, a conventional wireless finger-motion detecting apparatus has been developed, in which optical communication is performed between sensors and units receiving signals from the sensors. The conventional apparatus is disadvantageous in terms of use and cost. In addition to the inconvenience of installing the sensors on each finger, the conventional apparatus is expensive for it requires an additional battery for driving the sensors and an optical signal oscillator for oscillating optical signals.

### SUMMARY OF THE INVENTION

The present invention provides an apparatus and method for detecting finger-motion in a wireless manner through a small finger-motion detecting apparatus without an additional battery, which is inexpensive and convenient to use.

According to an aspect of the present invention, there is provided an apparatus for detecting finger-motion in a wireless manner comprising: a finger-motion signal receiving unit which outputs a wireless power signal, and receives and reads a wireless finger-motion signal to determine the corresponding finger-motion; a finger-motion signal transmitting unit, which generates a predetermined amount of power using the wireless power signal, receives a finger-motion signal corresponding to finger-motion using the predetermined amount of power, modulates the finger-motion signal into a finger-motion signal having a predetermined frequency, and outputs the modulated finger-motion signal in a wireless manner; and a finger-motion detecting unit, which determines whether or not finger-motion exists and generates the finger-motion signal corresponding to the finger-motion.

The finger-motion signal transmitting unit may include: a coil unit, which generates the predetermined amount of power using the wireless power signal and outputs the modulated finger-motion signal in a wireless manner; and a control unit, which is driven by the predetermined amount of power and is adapted to store a finger-motion signal inputted from the finger-motion detecting unit, and convert the finger-motion signal into the modulated finger-motion signal.

The coil unit may be wound about a finger, and the control unit may be positioned on top of the finger in the form of a chip.

According to another aspect of the present invention, there is provided a method for detecting finger-motion in a wireless manner comprising: (a) converting a predetermined wireless power signal into a predetermined amount of power; (b) detecting the motion of a user's finger using the predetermined amount of power, and generating a finger-motion signal corresponding to the finger-motion; (c) modulating the finger-motion signal into a finger-motion signal having a predetermined frequency, and outputting the modulated finger-motion signal in a wireless manner; and (d) receiving and reading the wireless finger-motion signal to determine which finger is moved.

The step (c) may include modulating the finger-motion signal into a finger-motion signal having a predetermined frequency depending on which finger is moved, and outputting the modulated finger-motion signal in a wireless manner.

The step (b) may include generating a finger-motion signal whenever the switch mounted on the user's finger is turned on.

An apparatus for detecting finger-motion according to preferred embodiments of the present invention will be explained hereinafter. Before that, radio frequency identification (RFID) technology, on which the present invention is based, needs to be explained in short. RFID systems which are widely used in contactless cards, such as transportation cards, include three elements, that is, an antenna, a transceiver which is often combined with a reader, and a tag which is called a transponder. The transponder is included in a card or the like, and is composed of an antenna formed out of a coil and an RFID chip which stores informnation.

During operation, the transceiver continuously transmits an electromagnetic wave through the antenna. When the transponder is in the range of an electromagnetic wave, the transponder generates an electromotive force according to Faraday's law to drive the RFID chip. The transponder transmits ID and data stored in the RFID chip to the transceiver through the antenna, which is connected to the RFID chip.

### BRIEF DESCRIPTION OF THE DRAWINGS

The above and other features and advantages of the present invention will become more apparent by describing in detail exemplary embodiments thereof with reference to the attached drawings in which:

FIGS. 1A and 1B are diagrams of a conventional wearable input device;

FIGS. 2A and 2B are diagrams of a finger-motion detecting apparatus according to a preferred embodiment of the present invention;

FIGS. 3A and 3B are diagrams of a finger-motion detecting apparatus according to a first preferred embodiment of the present invention, in which a finger-motion detecting unit is mounted on the end of a user's finger;

FIGS. 4A and 4B are diagrams of a finger-motion detecting apparatus according to a second preferred embodiment of the present invention, in which a finger-motion detecting unit is mounted on the distal joint of a user's finger;

FIG. 5 is a diagram of a finger-motion detecting apparatus according to a third preferred embodiment of the present invention, in which a finger-motion detecting unit is mounted on regions other than the end or the distal joint of a user's finger;

FIG. 6A is a schematic block diagram illustrating the configuration of the finger-motion detecting apparatus according to the present invention;

FIG. 6B is a detailed block diagram illustrating the configuration of the finger-motion detecting apparatus of FIG. 6A;

FIGS. 7A and 7B are circuit diagrams of the finger-motion detecting apparatus in FIGS. 6A and 6B; and

FIG. 8 is a flow chart illustrating a method for detecting finger-motion according to the present invention.

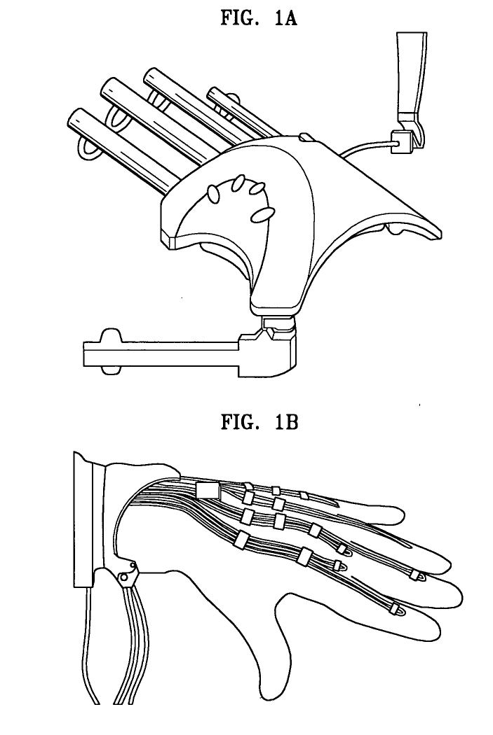

### DETAILED DESCRIPTION OF THE INVENTION

The present invention will now be described more fully with reference to the accompanying drawings, in which preferred embodiments of the invention are shown.

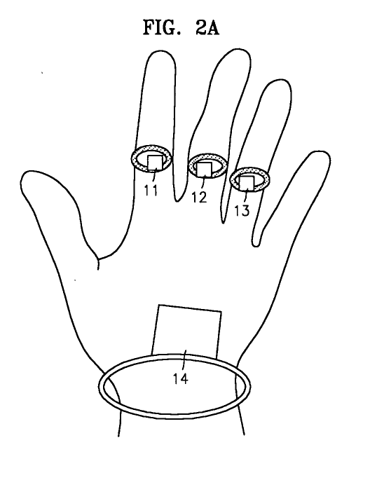

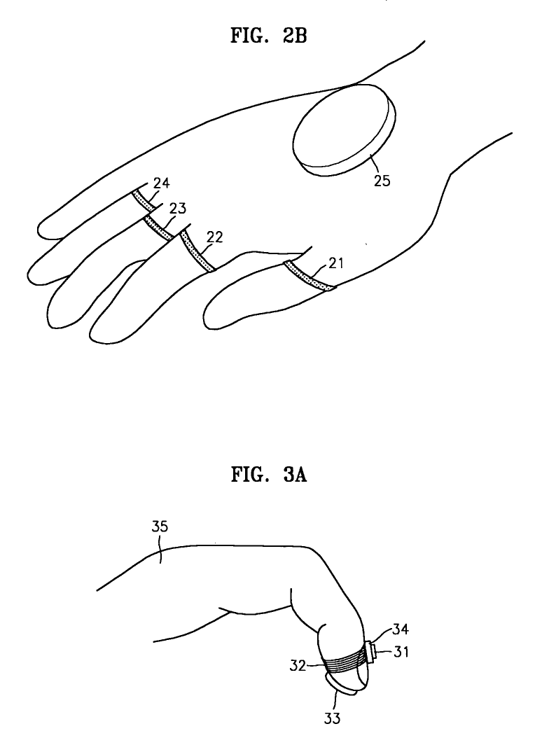

FIGS. 2A and 2B are diagrams of a finger-motion detecting apparatus according to a preferred embodiment of the present invention. Referring to FIGS. 2A and 2B, a finger-motion detecting apparatus of the present invention includes finger-motion detecting units 11 through 13 and 21 through 24 which are respectively installed on fingers and are adapted to detect finger-motion, and finger-motion signal receiving units 14 and 25 which receive in a wireless manner finger-motion information detected by the finger-motion detecting units 11 through 13 and 21 through 24. Thus, the finger-motion signal receiving units 14 and 25, in the finger-motion detecting apparatus of the present invention, correspond to an antenna and a transceiver of a radio frequency identification (RFID) system, and the finger-motion detecting units 11 through 13 and 21 through 24 correspond to a transponder of the RFID system.

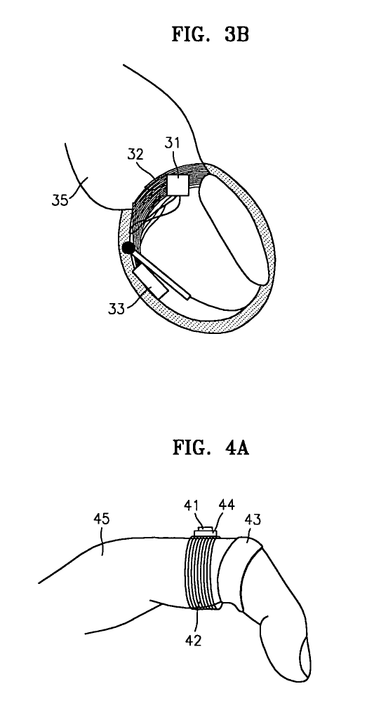

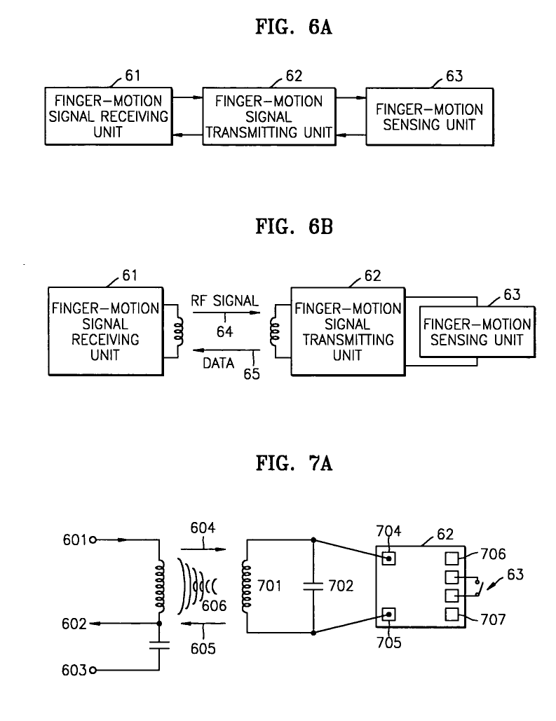

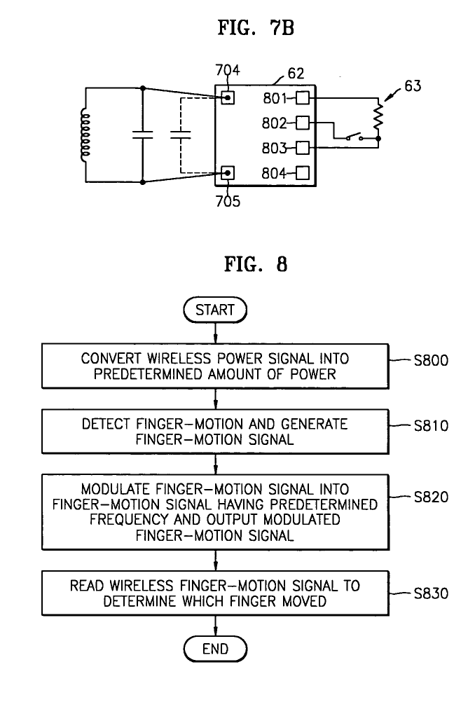

The internal structure of the finger-motion detecting apparatus according to the present invention will be explained later with reference to FIGS. 6A through 7B. The construction of the finger-motion detecting units will be explained first with reference to FIGS. 3A through 5.

FIGS. 3A and 3B are diagrams of finger-motion detecting units 11 through 13 and 21 through 24 according to a first preferred embodiment of the present invention. Each of the finger-motion detecting units includes a finger-motion sensing portion 33 which senses finger-motion and generates an electromagnetic signal, and a finger-motion signal transmitting unit which transmits in a wireless manner the finger-motion signal generated by the finger-motion sensing portion 33. Furthermore, the finger-motion signal transmitting unit includes a control unit 31 and a coil unit 32. The control unit 31 consists of a radio frequency identification (RFID) chip, which is driven using an electromotive force that is induced by an electromagnetic wave transmitted by the finger-motion signal receiving unit 14 or 25, and is adapted to store the finger-motion signal generated by the finger-motion sensing portion 33, and modulates the stored finger-motion signal into a finger-motion signal having a predetermined frequency, depending on which finger is moved. The coil unit 32 acts as an antenna which transmits the modulated signal to the finger-motion signal receiving unit 14 or 25.

The finger-motion detecting unit according to the first preferred embodiment shown in FIG. 3B is configured in the shape of a thimble. The finger-motion detecting unit includes a finger-motion sensing portion 33 which is disposed inside the finger-motion detecting unit for sensing finger-motion, a control unit 31 consisting of an RFID chip which stores a finger-motion signal inputted from the sensor and an ID of each finger, and modulates the finger-motion signal into a finger-motion signal having a predetermined frequency, a board 34 which helps affix the control unit 31 to the finger, and a coil unit 32 acting as an antenna, which is connected to the control unit 31, and is adapted to provide an induced electromotive force to the control unit 31 and transmit in a wireless manner the finger-motion signal modulated by the control unit 31 to the finger-motion signal receiving unit.

When a user puts the finger-motion detecting unit in the shape of a thimble on a specific finger or fingers and then taps on a floor with the finger or fingers, the finger-motion sensing portion 33 of the present invention senses the vibration of the finger or fingers and sends a finger-motion signal to the control unit 31 connected thereto. After receiving an electromotive force from the coil unit, the control unit 31 modulates a stored finger-motion signal into a finger-motion signal having a predetermined frequency, and outputs the modulated finger-motion signal to the finger-motion signal receiving unit 14 or 25 through the coil unit 32. The finger-motion sensing portion 33 depicted in FIG. 3A can be realized using various sensors, and most simply, using a switch. If the finger-motion sensing portion 33 is realized using a switch and the user taps on the floor with his or her finger or fingers, the switch located at the bottom of the finger-motion sensing portion is turned on and, accordingly, a finger-motion signal is transmitted to the control unit 31.

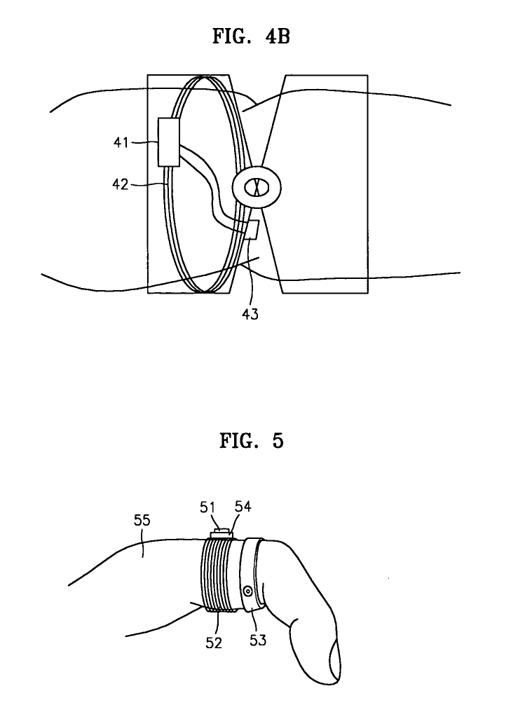

FIGS. 4A and 4B are diagrams of a finger-motion detecting apparatus according to a second preferred embodiment, in which a finger-motion detecting unit is installed on the distal joint of a user's finger.

Similar to the first preferred embodiment, each of the finger-motion detecting units according to the second preferred embodiment includes a finger-motion sensing portion 43 which senses finger-motion, a control unit 41 consisting of an RFID chip, which stores a finger-motion signal inputted from the sensing portion 43 and an ID of each finger and modulates the finger-motion signal into a finger-motion signal having a predetermined frequency, a board 44 which helps affix the control unit 41 to the finger, and a coil unit 42 acting as an antenna, which is connected to the control unit 41, and is adapted to provide an induced electromotive force to the control unit 41 and transmit in a wireless manner the finger-motion signal modulated by the control unit 41 to the finger-motion signal receiving unit 14 or 25 in a wireless manner.

When a user puts the finger-motion detecting apparatus on the distal joint of his or her specific finger or the distal joints of fingers, and flexes the joint or joints, the finger-motion sensing portion 43 of the present invention senses the finger-motion, and transmits a finger-motion signal to the control unit 41 connected thereto. After receiving an electromotive force from the coil unit 42, the control unit 41 modulates a stored finger-motion signal into a finger-motion signal having a predetermined frequency, and outputs the modulated signal to the finger-motion signal receiving unit 14 or 25 through the coil unit 42. The finger-motion sensing portion 43 depicted in FIG. 4A can be realized using various sensors, and most simply, using a switch. When the finger-motion sensing portion 43 is realized using a switch, the structure is divided into two parts which are connected in such a manner as to enable them to pivot along the direction in which the joint is moved, and the switch is mounted on a contact point between the two parts. When a user flexes the joint, the switch is turned on and accordingly a generated finger-motion signal is transmitted to the control unit 41.

FIG. 5 is a diagram of a finger-motion detecting apparatus according to a third preferred embodiment, in which a finger-motion detecting unit is installed on regions other than the end and the distal joint of a user's finger.

Similar to the first and second preferred embodiments, each of the finger-motion detecting units includes a finger-motion sensing portion 53 which senses finger-motion, a control unit 51 consisting of an RFID chip, a board 54 which helps affix the control unit 51 to the finger, and a coil unit 52 acting as an antenna, which is connected to the control unit 51, and is adapted to provide an electromotive force to the control unit 51 and transmit in a wireless manner a finger-motion signal modulated by the control unit 51 to the finger-motion signal receiving unit 14 or 25.

The finger-motion detecting apparatus according to the third preferred embodiment is different from the first and second preferred embodiments in that the finger-motion sensing portion is installed in such a manner as to protrude between adjacent fingers. That is to say, the sensor is interposed between two adjacent fingers, such that the finger-motion sensing portion generates a finger-motion signal when the adjacent fingers come in contact with each other. Further, when the finger-motion sensing portion of the third preferred embodiment is realized using a switch, it is also possible to generate a finger-motion signal by directly pressing the switch interposed between the adjacent fingers using the thumb.

The internal structure of the finger-motion detecting apparatus of the present invention will be explained with reference to FIGS. 6A through 7B.

FIG. 6A is a schematic block diagram illustrating the configuration of the finger-motion detecting apparatus according to the present invention.

Referring to FIG. 6A, the finger-motion detecting apparatus includes a finger-motion signal receiving unit 61 which outputs a wireless power signal, and receives and reads a wireless finger-motion signal to determine the corresponding finger-motion, a finger-motion signal transmitting unit 62, which generates a predetermined amount of power using the wireless power signal, receives a finger-motion signal corresponding to the finger-motion using the predetermined amount of power, converts the finger-motion signal into a finger-motion signal having a predetermined frequency, and outputs the finger-motion signal having the predetermined frequency in a wireless manner, and a finger-motion sensing portion 63, which senses whether or not finger-motion exists and generates a finger-motion signal corresponding to the finger-motion. FIG. 6B is a detailed block diagram illustrating the configuration of the finger-motion detecting apparatus of FIG. 6A.

FIG. 7A is a circuit diagram of the finger-motion detecting apparatus in FIGS. 6A and 6B. FIG. 8 is a flow chart illustrating a method for detecting finger-motion. Referring to FIG. 7A and FIG. 8, in step S800, the finger-motion signal receiving unit 61 generates an electromagnetic wave 606 which passes through a coil by using a predetermined alternating current power to transmit a wireless power signal. A capacitor 702 rectifies an electromotive force induced from a coil unit 701 of the finger-motion signal transmitting unit 62 and transmits the rectified electromotive force to the RFID chip to drive the RFID chip.

In step S810, the RFID chip receives and stores finger-motion generation information through the finger-motion sensing portion 63, which is realized using a switch, connected to terminals 706 and 707 and generates a finger-motion signal 605, which includes the finger-motion generation information and an ID of each finger. Next, in step S820, the RFID chip modulates the finger-motion signal 605 into a finger-motion signal having a predetermined frequency and outputs the modulated finger-motion signal through the coil unit 701.

In step S830, the finger-motion signal receiving unit 61 installed on the back of the user's hand receives a wireless finger-motion signal through an antenna represented by the coil 602 and reads the received finger-motion signal to determine which finger is moved. Meanwhile, FIG. 7B is a circuit diagram illustrating another configuration of the finger-motion signal transmitting unit 62 and the finger-motion sensing portion 63 of the finger-motion detecting apparatus.

The apparatus and method for detecting finger-motion according to preferred embodiments of the present invention has been explained. The finger-motion detecting apparatus of the present invention can be used by itself or in combination with a virtual input device, such as a virtual keyboard or a virtual mouse.

For example, when the finger-motion detecting apparatus of the present invention is used in combination with a virtual mouse, an inertial sensor is mounted on the aforementioned finger-motion signal receiving unit. The inertial sensor senses finger-motion using an output of the inertial sensor and determines the position of the mouse pointer. According to the present invention, a virtual mouse can be realized by using the motion signal of a predetermined finger as a click signal of the mouse, e.g., the motion of a user's index finger is set to the left button click of the mouse, and the motion of the user's middle finger is set to the right button click of the mouse.

On the other hand, when a virtual keyboard is realized using the finger-motion detecting apparatus of the present invention, a virtual keyboard having a shape similar to a QWERTY keyboard is output on a computer monitor or a display of a specific device, such as a personal digital assistant (PDA). Next, an inertial sensor, such as a gyroscope, is mounted on the finger-motion signal receiving unit to measure a position in a space. When a user wears the finger-motion detecting apparatus of the present invention on his or her hand and moves his or her hand, the position of the whole hand and the positions of fingers are displayed in real-time on the virtual keyboard by virtue of the inertial sensor. When the user moves his or her finger to a specific position, the generated finger-motion signal of the present invention is interpreted as a keystroke on the virtual keyboard, such that the detected position of the finger corresponds to a key on the virtual keyboard, and the letter assigned to the key is inputted.

As described above, the present invention connects a body to a finger-motion signal input unit that operates in a wireless manner, in contrast to a conventional space-based keyboard or a conventional data glove-type input device. Therefore, a user can use his or her hand more freely, and compared with the prior art, it becomes easier to wear the apparatus. Also, since a small wireless module using RFID technology is employed without an additional battery, it is easy to carry the apparatus and the number of usable sensors can be freely changed.

While the present invention has been particularly shown and described with reference to exemplary embodiments thereof, it will be understood by those of ordinary skill in the art that various changes in form and details may be made therein without departing from the spirit and scope of the present invention as defined by the following claims.

What is claimed is:

1. An apparatus for detecting finger-motion in a wireless manner comprising:
a finger-motion signal receiving unit, which outputs a wireless power signal and receives and reads a wireless finger-motion signal corresponding to finger-motion to detect which finger is moved;
a finger-motion signal transmitting unit, which generates a predetermined amount of power using the wireless power signal, receives a finger-motion signal corresponding to finger-motion using the predetermined amount of power, modulates the finger-motion signal into a finger-motion signal having a predetermined frequency, and outputs the modulated finger-motion signal in a wireless manner; and
a finger-motion detecting unit, which determines whether or not finger-motion exists and generates the finger-motion signal corresponding to the finger-motion.

2. The apparatus of claim 1, wherein the finger-motion signal transmitting unit includes:
a coil unit which generates the predetermined amount of power using the wireless power signal, and outputs the modulated finger-motion signal in a wireless manner; and
a control unit which is driven by the predetermined amount of power, is adapted to store the finger-motion signal inputted from the finger-motion detecting unit, and converts the finger-motion signal into the modulated finger-motion signal.

3. The apparatus of claim 2, wherein the control unit converts an alternating current power generated by the coil unit into a direct current power to generate the predetermined amount of power.

4. The apparatus of claim 2, wherein the control unit modulates the finger-motion signal into the finger-motion signal having a predetermined frequency, depending on which finger is moved, and outputs the modulated finger-motion signal.

5. The apparatus of claim 2, wherein the coil unit is wound about a finger whose motion is to be detected, and the control unit is positioned on top of the finger in the form of a chip.

6. The apparatus of claim 1, wherein the finger-motion detecting unit is configured in the form of a switch, and is adapted to generate the finger-motion signal when the switch is turned on.

7. The apparatus of claim 6, wherein the switch is mounted on a predetermined joint of a user's finger, and is adapted to generate the finger-motion signal when the switch is turned on by user flexing a joint.

8. The apparatus of claim 6, wherein the switch is mounted on an end of a user's finger, and is adapted to generate the finger-motion signal when the switch is turned on by a user tapping with the finger.

9. The apparatus of claim 6, wherein the switch is installed between a user's adjacent fingers, and is adapted to generate the finger-motion signal when a first finger, on which the switch is installed, and a second finger, adjacent to the first finger, come in contact with each other and the switch is turned on.

10. The apparatus of claim 6, wherein the switch is installed on a user's finger, and is adapted to generate the finger-motion signal when the finger, on which the switch is installed, and the thumb come in contact with each other and the switch is turned on.

11. A method for detecting finger-motion in a wireless manner comprising:
(a) converting a predetermined wireless power signal into a predetermined amount of power;
(b) detecting a motion of a user's finger using the predetermined amount of power and generating a finger-motion signal corresponding to the finger-motion;
(c) modulating the finger-motion signal into a finger-motion signal having a predetermined frequency and outputting the modulated finger-motion signal in a wireless manner; and
(d) receiving and reading the wireless finger-motion signal having the predetermined frequency and determining which finger is moved.

12. The method of claim 11, wherein the step (a) comprises converting an alternating current power induced by the wireless power signal into the predetermined amount of power by rectifying the alternating current power.

13. The method of claim 11, wherein the step (c) comprises modulating the finger-motion signal into the finger-motion signal having a predetermined frequency, depending on which finger is moved, and outputting the modulated finger-motion signal in the wireless manner.

14. The method of claim11, wherein the step (b) comprises generating the finger-motion signal when a switch installed on the user's finger is turned on.

15. The method of claim 14, wherein the switch is mounted on a predetermined joint of the user's finger, and is adapted to generate a finger-motion signal when the switch is turned on by a user flexing a joint.

16. The method of claim 14, wherein the switch is mounted on an end of the user's finger, and is adapted to generate a finger-motion signal when the switch is turned on by a user tapping on a surface with the finger.

17. The method of claim 14, wherein the switch is mounted between adjacent fingers, and is adapted to generate the finger-motion signal when a first finger, on which the switch is mounted, and a second finger, adjacent to the first finger, come in contact with each other and the switch is turned on.

18. The method of claim 14, wherein the switch is mounted on the user's finger, and is adapted to generate the finger-motion signal when the finger, on which the switch is mounted, and a thumb come in contact with each other and the switch is turned on.
